(19) United States
(12) Patent Application Publication
(10) Pub. No.: US 2025/0057415 A1
(43) Pub. Date: Feb. 20, 2025

(54) APPARATUS, METHOD AND SYSTEM FOR EVALUATING EXOPHTHALMIA IN EXOPHTHALMIA TESTING

(71) Applicant: SHANGHAI BAIYI HEALTHCARE TECHNOLOGY CO., LTD ., Shanghai (CN)

(72) Inventors: Chaonan TIAN, Shanghai (CN); Youxiang WANG, Shanghai (CN); Youzhi WANG, Shanghai (CN); Dong DU, Shanghai (CN)

(21) Appl. No.: 18/932,903

(22) Filed: Oct. 31, 2024

Related U.S. Application Data
(63) Continuation of application No. PCT/CN2022/142941, filed on Dec. 28, 2022.

(30) Foreign Application Priority Data
Aug. 18, 2022 (CN) 202210989502.6

(51) Int. Cl. A61B 3/107; A61B 3/00; A61B 3/14

(52) U.S. Cl. CPC A61B 3/107; A61B 3/0025; A61B 3/145

(57) ABSTRACT

A device for evaluating exophthalmia includes a left displacement platform, a left canthal latch point, a left mirror, a camera, a near infrared light source, a right canthal latch point, a right mirror, a right displacement platform, a visible light column and a shell or housing. A method for evaluating exophthalmia includes positioning the outer canthus of the eye, emitting light from the near-infrared light source and successively emitting light from multiple visible light columns, recording video of the eyeball looking straight ahead through the camera, and obtaining the video frame of the longest reflected light of the visible light columns on the eyeball in the mirror from the video; The corneal vertex is determined from the video frame, and the pupil center is determined by a neural network. The exophthalmia is calculated from known positions and the mirror tilt angle, the corneal vertex, and the pupil center.

### CROSS-REFERENCE TO RELATED APPLICATIONS

This application is a continuation of International Pat. Appl. No. PCT/CN2022/142941, filed on Dec. 28, 2022, which claims priority to Chinese Pat. Appl. No. 202210989502.6, filed on Aug. 18, 2022, the contents of each of which are incorporated by reference herein in their entireties.

### TECHNICAL FIELD

The invention relates to the technical field of eye examination and testing, in particular to a device, method and system for evaluating exophthalmia in exophthalmia testing.

### BACKGROUND

Exophthalmia refers to the distance from the vertex of the cornea of the eyeball to the temporal orbital margin. The average ocular exophthalmia of normal people is 12-14 mm, the average is 13 mm, and the difference between the two eyes is not more than 2 mm. However, patients with orbital diseases often have increased ocular exophthalmia. At present, Hertel ophthalmoscopes and CT scanning are the main methods for measuring exophthalmia in clinics.

A Hertel exophthalmia meter is a contact measurement, which requires manual recording of the degree during the experiment. The evaluation results between individuals are closely related to the experience of the diagnostic physician and the measurement method used, which is easy to lead to individual differences. Moreover, the close contact between patients and doctors in the clinical diagnosis and treatment environment increases the risk of infection exposure of both subjects and testers. In addition, some studies have suggested that when using Hertel exophthalmia tester for detection, because the transverse orbital edge is not completely symmetric, the degree of soft tissue collusion may vary, and there are no unified application standards or operating rules relating to the visual error and experience of the examiner, which will lead to significant differences in the readings of different examiners. Testers with significant experience tend to read 1 mm larger than inexperienced observers. Although the measurement accuracy of CT scans are higher than that in Hertel exophthalmia testing, there are problems such as large radiation doses, high measurement costs, and slow result generation.

In view of the problems of low accuracy and high cost of the above methods, there are also solutions that use automatic measuring equipment and methods to determine the exophthalmia. For example, as disclosed by Chinese patent no. CN104720738A (application number: 201510155640.4, publication date: Jun. 24, 2015), a first distance X1 from a system reference point St of the ophthalmic device to the lateral frame edge Ek of the human eye in the front and back directions, a second distance X2 from the system probe of the ophthalmic device to the system reference point St in the front and back directions, and a third distance X3 from the corneal vertex Ec to the system probe in the front and back directions are obtained. The exophthalmia was obtained using the formula X1+X2−X3. As described in Chinese patent no. CN112806956A (application number: 202110139034.9, public date: May 18, 2021), a range sensor is moved on a driver to identify the corneal vertex, and the distance between the light emitter and the range sensor is calculated to obtain the ocular prominence. The above methods have the disadvantages of complex equipment and inaccurate corneal vertex recognition.

### SUMMARY

In order to overcome the problems of large errors and high costs in the measurement of exophthalmia in the prior art, the invention provides an exophthalmia evaluation device, method and system for exophthalmia examination, aiming at improving the accuracy of the measurement of exophthalmia.

A first aspect of the invention provides an exophthalmia evaluation device (e.g., an exophthalmometer), which comprises a left displacement platform, a left canthal latch point, a left mirror, one or more cameras, a near infrared light source, a right canthal latch point, a right mirror, a right displacement platform, a visible light column and a shell or housing. The left mirror and the right mirror are respectively on the left canthal latch point and the right canthal latch point. The left canthal latch point and the right canthal latch point are respectively on the left displacement platform and the right displacement platform. The left displacement platform and the right displacement platform are respectively on opposite sides of the shell or housing, and the left mirror and the right mirror are angled. The camera(s) are in front of the left mirror and the right mirror. The near infrared light source is below the camera(s). The visible light column comprises multiple columns configured longitudinally inside (e.g., along the inner surface) of the shell or housing.

Further, the device may include two cameras, a first one in front of the left mirror and a second one in front of the right mirror.

Further, each of the visible light columns may include one or more discrete point sources of light.

The device may also further comprise a jaw support and a head rest (e.g., a forehead bracket), either or both of which may have an adjustable position. Each of the jaw support and the head rest may be configured to place the eye (or, more specifically, the outer canthus) close to (e.g., in the proximity of) an end of at least one of the left and right canthal latch points. The end of the canthal latch point(s) proximate to the eye may be an innermost end, on which the corresponding mirror may be fixed or mounted.

The left displacement platform and the right displacement platform may be adjustable along one or more Cartesian dimensions, and thus be configured to position a corresponding canthal latch point and/or mirror relative to the eye to be evaluated. Alternatively, or in addition, the left mirror and the right mirror may be respectively fixed to the left canthal latch point and the right canthal latch point, and the left canthal latch point and the right canthal latch point may rotate angularly or move in one or more Cartesian dimensions to position the left mirror and the right mirror relative to the outer canthus of the corresponding eye. The mirrors may be configured to rotate on the corresponding canthal latch point in one or more angular dimensions, so that the angle of the mirrors relative to the plane of the outer canthus can be adjusted.

A second aspect of the invention provides a method for evaluating exophthalmia, which may be based on the exophthalmia evaluation device described in the first aspect of the invention, and which comprises positioning an outer canthus of an eye, emitting light from the near-infrared light source and (e.g., while) emitting light successively from multiple visible light columns, recording a video of an eyeball of the eye looking straight ahead using a camera, obtaining from the video the video frame having the longest reflected light from the visible light columns appearing on the eyeball in a mirror, determining a corneal vertex (e.g., of the eyeball) from the video frame and determining the pupil center (e.g., of the eyeball) using a neural network, and calculating the exophthalmia from known positions and a tilt angle of the mirror, the corneal vertex, and the pupil center.

The known positions include the position of the optical center of the camera, the distance between the optical center of the camera and the video frame, and the position of the outer canthus of the eye. The tilt angle of the mirror is the angle between the mirror and the imaging surface of the camera.

Furthermore, the multiple visible light columns may be illuminated successively from the middle to the outside (e.g., of a shell or housing in which the multiple visible light columns are arranged longitudinally, or of a 1-by-n array of the visible light columns, where n is a positive integer and is the number of the visible light columns), or from the outside to the middle.

Furthermore, the video may be captured of the eyeball on one side (e.g., of the shell or housing, or the head or face containing the eyeball positioned against or in the shell or housing) when the visible light columns on an opposite side (e.g., the other side of the shell or housing containing the visible light columns and in or against which the head or face is positioned) emit light. Alternatively, the video of the other eye is taken when the visible light columns on one side (e.g., opposite from that of the other eye) emit light.

Furthermore, when calculating the exophthalmia, the optical center (or its position) may be defined as O, the outer canthus of the eye (or its position) may be defined as E, the distance (or a corresponding line or line segment) between the optical center O and the video frame may be defined as OB, the corneal vertex (or its position) may be defined as Z, the imaging point of the corneal vertex on the video frame may be defined as imaging point C, a reflection point of the corneal vertex in the mirror (or its position) may be defined as U, and the imaging point of the reflection point U on the video frame may be defined as imaging point A. The vertical distance (or a corresponding line or line segment) between the imaging point C and the optical axis may be defined as AB, and the vertical distance (or a corresponding line or line segment) between the imaging point A and the optical axis may be defined as CB. The tilt angle (e.g., between the mirror and the imaging surface of the camera) may be defined as ∠UED. The distance (or a corresponding line or line segment) between the optical center and the plane containing the outer canthus may be defined as OF. The distance (or a corresponding line or line segment) between the outer canthus and the optical axis may be defined as EF. The exophthalmia (or its numerical value, which may be the distance from the plane containing the outer canthus to the corneal vertex) may be defined as ZG. ZG may also be defined as the vertical or orthogonal distance between the corneal vertex Z and a straight line DE in the plane containing the outer canthus. In addition, the plane containing the outer canthus may also include the pupil center or a location thereof that may be defined as a point G. Each of the lengths of OB, OF, EF, AB, CB and the tilt angle ∠UED is known.

The exophthalmia (or its numerical value) may be calculated by calculating (e.g., generating) a line that passes through the reflection point U of the corneal vertex Z in the mirror and is parallel to the straight line DE that intersects with the optical axis at a point W, with ZG at a point Y, and with an extension (e.g., a linear extension) of a line OC (i.e., a line between the optical center O and the imaging point C) at a point X; determining the length of a line segment WF (i.e., a line between the point W and a point F on the straight line DE that is parallel to a line or line segment corresponding to or representing ZG) according to:

$$WF = \frac{OF tan ∠ UOW - EF}{tan ∠ UOW + \frac{1}{tan ∠ UED}}$$

where <UOW is an angle between (i) a line from the reflection point U to the optical center O (e.g., a line or line segment UO) and (ii) the optical axis (e.g., a line or line segment OW, between the optical center O and the point W); iteratively calculating a length of a line segment ZY (i.e., between the corneal vertex and the point Y); and calculating the exophthalmia according to ZG=WF+ZY.

Positioning the outer canthus of the eye may comprise positioning the head containing the eye in a jaw support and a head rest (each of which may be adjustable). Each of the jaw support and the head rest may be independently configured to place the eye (or its outer canthus) in proximity to an end of a canthal latch point to which the mirror is fixed or mounted. The method may further comprise adjusting a position of the displacement platform and/or the canthal latch point to bring the mirror to a predetermined position relative to the position of the outer canthus.

According to another aspect of the invention, a system for evaluating exophthalmia (which may implementing the method as in the second aspect of the invention and which may operate in conjunction with the device as in the first aspect of the invention) comprises a video acquisition module, configured to acquire the video of the eyeball looking straight ahead when the near infrared light source emits light and the multiple visible light columns successively emit light; an image acquisition module, configured to obtain from the video the video frame having the longest reflected light in the mirror; a feature extraction module, configured to extract the corneal vertex and the pupil center from the video frame; and a calculation module, configured to calculate the exophthalmia from the known positions and the mirror tilt angle, the corneal vertex, and the pupil center. As in the other aspects of the invention, the known positions include the position of the optical center of the camera, the distance between the optical center of the camera and the video frame, and the position of the outer canthus of the corner of the eye. Also, the tilt angle of the mirror is the angle between the mirror and the imaging surface, as in the other aspects of the invention.

According to yet another aspect of the invention, a tangible computer-readable storage medium storing at least one program code adapted to be loaded and executed by a processor may perform the method of evaluating exophthalmia as in the second aspect of the invention.

Compared with the prior art, the technical scheme of the invention has at least the following beneficial effects:

The eye video is captured in the near infrared light field by the evaluation device as visible light from the visible light columns is reflected from the surface of the eyeball at different angles and/or at different focal points, to accurately identify the corneal vertex of the eye. The iris, pupil and sclera in the video are effectively distinguished using a neural network, so as to accurately identify the pupil center. The longitudinal visible light columns arranged around the face are successively lit to find the longest light from the eyeball in the mirror. The “longest light,” “longest reflected light,” and grammatical variations thereof generally (but not necessarily, depending on the context of its use) refer to the light from a visible light column that is reflected by the cornea to the mirror and that provides the largest value for the exophthalmia. The corneal vertex can be accurately and easily identified using this longest reflected light. After identifying the center of the pupil and the vertex of the cornea, combining certain known positions (e.g., of structures in the camera and/or eye) and the tilt angle of the mirror, the invention can accurately calculate exophthalmia (and, optionally, the prominence of the eyeball) using optical imaging principles without touching the cornea, so as to ensure accuracy and safety in the exophthalmia measurement.

### BRIEF DESCRIPTION OF THE DRAWINGS

FIG. 1 is a stereogram of an exemplary exophthalmia evaluation device for exophthalmia testing in accordance with the invention.

FIG. 2 is a flow chart of an exemplary method of evaluating exophthalmia in accordance with the invention.

FIG. 3 shows an exemplary video frame taken when the light reflected in the mirror is at its longest.

FIG. 4 is a diagram showing one or more exemplary bases and/or principles for calculating exophthalmia.

FIG. 5 is a diagram showing an exemplary system for evaluating exophthalmia.

In the Figures, the following identification numbers refer to the corresponding structures and/or features: 1—Lower support frame; 2—Jaw bracket or support; 3—left displacement platform; 4—Left canthal latch point; 5—Left mirror; 6—headrest or head bracket; 7—Upper support frame; 8—Camera; 9—Near infrared light source; 10—Right canthal latch point; 11—Right mirror; 12—right displacement platform; 13—visible light column; 14—Shell; 501—Video capture module; 502—Image acquisition module; 503—Feature extraction module; 504—Computing module.

### DETAILED DESCRIPTION OF THE PREFERRED EMBODIMENTS

Specific embodiments of the invention are further described in detail in the examples below, in combination with the attached drawings. The following embodiments are used to illustrate the invention, but not to limit the scope of the invention.

It should be noted that OB, AB, CB, EF, etc. used in calculations in the invention refer to a corresponding line segment or its length.

### Example 1

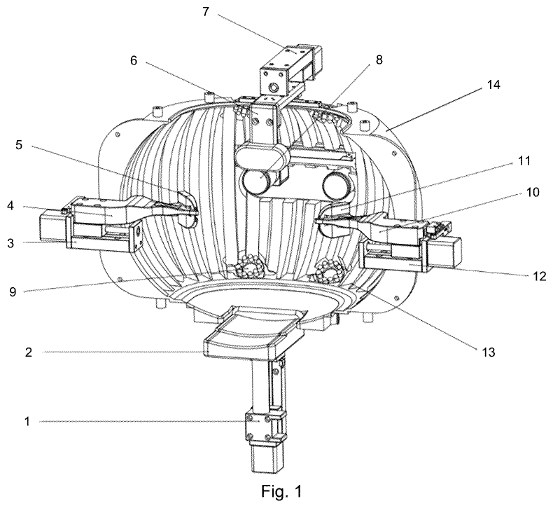

The example and/or embodiment shown in FIG. 1 provides an exophthalmia evaluation device for exophthalmia testing, including a left displacement platform (3), a left canthal latch point (4), a left mirror (5), cameras (8), a near-infrared light source (9), a right canthal latch point (10), a right mirror (11), a right displacement platform (12), a plurality of visible light columns (13), and a shell or housing (14). The left mirror (5) and the right mirror (11) are respectively configured on the left canthal latch point (4) and the right canthal latch point (10), and the left canthal latch point (4) and the right canthal latch point (10) are respectively on the left displacement platform (3) and the right displacement platform (12). The left displacement platform (3) and the right displacement platform (12) are respectively on opposite sides of the shell or housing (14), and are configured to adjust a position of the corresponding left and right mirrors relative to the outer canthus of the eye to be evaluated or tested. The left mirror (5) and the right mirror (11) are angled, e.g. at an angle adapted to receive light from the visible light columns reflected from the surface of the eyeball, and further reflect the light reflected from the surface of the eyeball to an imaging device in the corresponding camera. The cameras (8) are thus configured in front of the left mirror (5) and the right mirror (11), to receive light reflected by the left mirror (5) and the right mirror (11). The near infrared light source (9, of which there may be two) is configured below the cameras (8), and when there are two near infrared light sources (9), they may be spaced apart by a same distance as the cameras (8), along a line that is parallel to a line through the center of each camera (8). The visible light columns (13) are configured longitudinally inside (e.g., along an inner surface of) the shell or housing (14).

In order to further support and adjust the evaluation device and to ensure that the evaluation device is positioned correctly relative to the eye(s) to be evaluated, the evaluation device also includes a lower support frame 1, a jaw bracket 2, a forehead bracket 6 and an upper support frame 7. The lower support frame 1 and the upper support frame 7 are configured at the lower and upper parts of the shell 14, respectively, to support and rotate the entire evaluation device, and the jaw bracket 2 and the forehead bracket 6 are configured to secure the head or face containing the eye(s) to be evaluated in a fixed position. In some embodiments, the positions of the jaw bracket 2 and the forehead bracket 6 are independently adjustable (e.g., in and/or along one or more Cartesian dimensions).

Further, there can be two cameras 8, one in front of the left mirror 5 and the other in front of the right mirror 11, to take videos of the left and right eyes, respectively. In order to provide a uniform near-infrared light field, the near-infrared light source 9 can include multiple light sources, evenly distributed under the camera 8. Each longitudinal visible light column 13 can be configured as an array of point-like light sources, and may be externally wrapped in silicone.

When the device is used to measure exophthalmia or ocular prominence, the left displacement platform 3 and the right displacement platform 12 are first adjusted (e.g., along one or more Cartesian dimensions, to position the corresponding canthal latch point and/or mirror as described herein), and the left mirror (5) and the right mirror (11) are fixed in position relative to the outer canthus of the corresponding eye of the user (e.g., the eye to be evaluated) using the left canthal latch point (4) and the right canthal latch point (10) (which may rotate angularly and/or move in one or more Cartesian dimensions), to adapt to the facial features of different persons. After fixing the user's eyes and face in position, the near-infrared light source 9 is turned on, and a series of visible light columns 13 configured vertically are turned on from the middle to the outside (e.g., of the shell), or from the outside to the middle. The visible light columns 13 successively emit light of different lengths and/or angles to the eyeball, which reflects the light to the mirror. The video frame having the longest reflected light (e.g., in which the visible light column 13 forms the longest projection on the cornea through the corneal vertex) can be found in the video taken. Through the video frame taken in the light field of the near infrared light source 9, the pupil center can be accurately found by neural network analysis. The corneal vertex and pupil center, combined with the known distance and the mirror tilt angle, are used to calculate the exophthalmia.

### Example 2

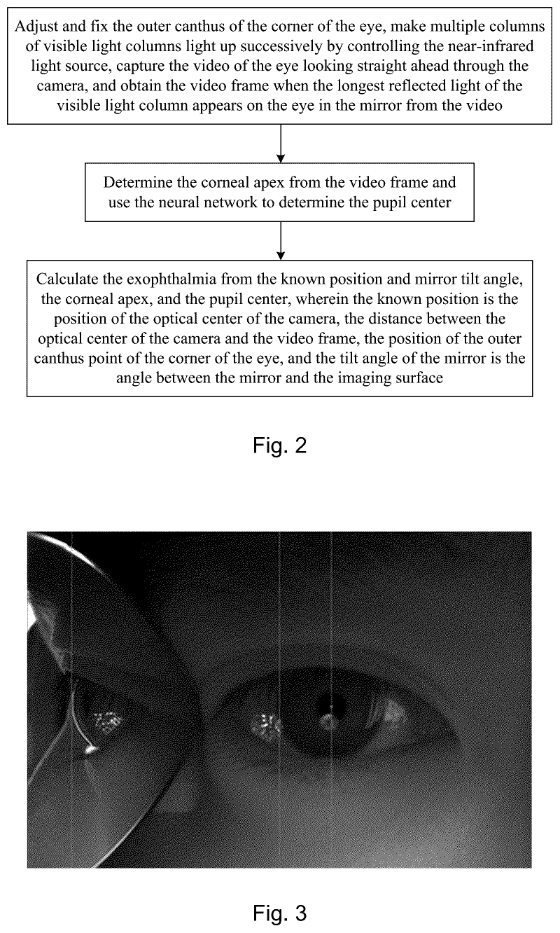

As shown in FIGS. 2 and 3, this example provides a method for evaluating exophthalmia, which is illustrated below in conjunction with an exophthalmia evaluation device for exophthalmia testing. The evaluation of exophthalmia involves the following steps:

Step S1: Adjust and fix the outer canthus of the corner of the eye, make multiple visible light columns light up successively and control the near-infrared light source, capture the video of the eyeball looking straight ahead through the camera, and obtain the video frame of the longest reflected light of the visible light columns on the eyeball in the mirror from the video.

In Step S1 above, when the longitudinal visible light column on one side emits light, the video of the opposite eye is taken. Specifically, when a left visible light column 13 lights up, the video of the right eye is taken, and when a right visible light column 13 lights up, the video of the left eye is taken. Due to the setup of a series of longitudinal visible light columns 13, and the left mirror 5 and the right mirror 11 tilt angles, the visible light columns 13 are turned on successively from the outside to the inside, so that the visible light columns 13 present a series of reflected light of different lengths on the side of the eye in the mirror, and the longest projection of light from the visible light columns 13 on the cornea can be found through the reflected light on the eye in the mirror. For example, when the rightmost visible light column 13 emits light, the light reflected from the left eye on the side of the eye in the left mirror 5 is close to the inner canthus of the eye. As the light is emitted successively from visible light columns 13 towards the middle, the light from the light column 13 reflected in the side of the eye in the left mirror 5 gradually becomes longer until the reflected light passes through the vertex of the cornea, at which time the reflected light is the longest. The video frame at this point is obtained from the video.

Step S2: Determine the corneal vertex from the video frame and use the neural network to determine the pupil center.

As shown in FIG. 3, the corneal vertex can be determined by the video frame having the longest reflected light from the eyeball in the mirror. The corneal vertex is the tangent point of the reflected light on the eyeball in the left mirror and the vertical line in FIG. 3. In the normal visible light band of 400-700 nm, the color of different parts of the eye, namely the pupil, iris, and sclera, has little effect on imaging, due to the gradual structure of the corneal limbus in the contact part of the iris and sclera, resulting in the inability to accurately identify the spherical center of the eye. The peak absorption of human melanin pigment occurs at about 335 nm, and it is almost completely non-absorbed for wavelengths over 700 nm, while the reflectivity of iris is quite stable in near-infrared wavelengths over 700 nm. Therefore, the invention adopts a near-infrared light field, which can clearly distinguish the sclera, iris and pupil boundaries, and can accurately identify the pupil center in combination with a neural network training model. In one implementation, the RITnet neural network model is used in this example. Pre-trained models and source code for RITnet-based neural networks are available at bitbucket.org/eye-ush/ritnet/(e.g., bitbucket.org/eye-ush/ritnet/src/master/).

The exophthalmia evaluation device provided in Example 1 for exophthalmia testing also provides the ability to determine the pupillary center by manual operation after determining the corneal vertex from the video frame, which the user can select on demand with the neural network or by manual operation.

Step S3: Calculate the exophthalmia (proptosis of the eyeball) based on the known position and tilt angle of the mirror, the corneal vertex, and the pupil center.

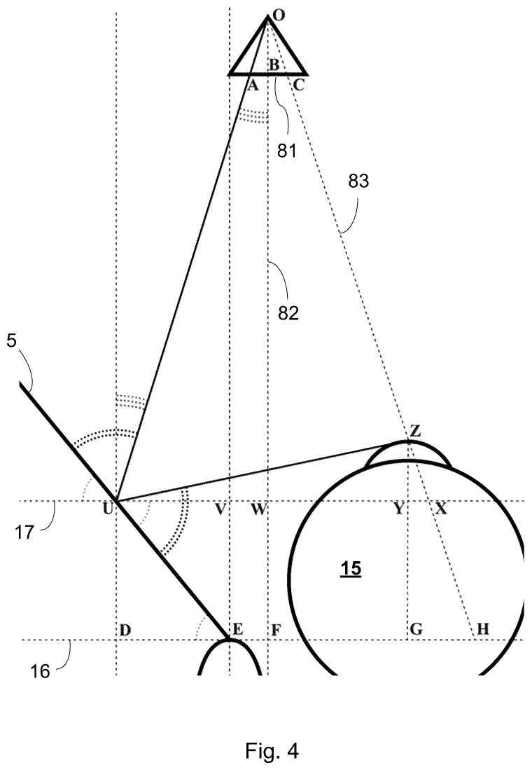

In step S3, the known positions include the position of the optical center of the camera (e.g., point O in FIG. 4), the distance between the optical center of the camera and the video frame (e . . . , the length of the line segment OB in FIG. 4), and the position of the outer canthus of the corner of the eye (e.g., point E in FIG. 4), although other known positions (e.g., equivalent to the stated known positions) may also be used when calculating the exophthalmia of an eye. The tilt angle of the mirror is the angle between the mirror and the imaging surface of the camera. The exophthalmic distance can be obtained through the known positions, which include the distance between the optical center of the camera and the imaging surface (that is, the photosensor or imaging sensor), which is called the image distance in photography. Through the above-mentioned distance, the mirror tilt angle, the calculated location (e.g., an imaging point) of the pupil center, and the corneal vertex on the video frame, the exophthalmia (e.g., its value, distance or length) can be calculated.

### Example 3

Based on Example 2 above, this Example 3 further explains how to perform the mathematical calculation of exophthalmia (e.g., (the exophthalmic distance).

As shown in FIG. 4, the optical center is O, the outer canthus is E, the vertical distance between the optical center O and the video frame 81 is OB, the imaging point of the corneal vertex Z on the video frame 81 is imaging point C, the imaging point of the reflection point U of the corneal vertex Z in the mirror 5 on the video frame 81 is the imaging point A, the distance between imaging point C and the optical axis 82 is CB, and the distance between imaging point A and the optical axis 82 is AB. The angle of inclination between the mirror 5 and the eye 15 (e.g., line or line segment DE) is ∠UED, the distance between the optical center O and the face plane 16 including the outer canthus E is OF, the distance between the outer canthus E and the optical axis 82 is EF, and the exophthalmia is the vertical distance ZG from the corneal vertex Z to the point G on the straight line DE. The point G may, in some embodiments, be the location of the pupil center, determined using the neural network, and in other embodiments, it may be a reference location in the eyeball, at the point where the line or line segment ZG intersects the face plane 16. The line or line segment ZG may be perpendicular or normal to the face plane 16. The lengths of OB, OF, EF, AB and CB and the angle ∠UED are known.

The exophthalmia may be calculated as follows.

A line 17 that passes through the reflection point U of the corneal vertex Z in the mirror 5 and is parallel to the line DE may be generated or calculated that intersects with the optical axis at point W, with ZG at point Y, and with an extension 83 of the line OC at point X.

According to principles of imaging, both the imaging point C of the corneal vertex Z and the imaging point A of the reflection point U of the corneal vertex Z in the mirror 5 are in the video frame 81. The distance CB and AB of imaging point C and imaging point A from the optical axis 82 can be read in the video frame 81. According to principles of light reflection, the incidence angle of the corneal vertex Z in the mirror 5 is equal to the reflection angle, namely:

$$∠ UOW + ( \frac{Π}{2} - ∠ UED ) = ∠ ZUY + ∠ UED$$

This leads to:

$$∠ ZUY = \frac{Π}{2} + ∠ UOW - 2 ∠ UED$$

Given the known or calculatable lengths of OB, OF, AB, CB and EF, and the calculatable size of ∠UED, the following can be obtained according to analytic geometry:

$$tan ∠ UOW = \frac{AB}{OB} \qquad ( 1 )$$

$$tan ∠ WOX = \frac{CB}{OB} \qquad ( 2 )$$

$$\{ \begin{matrix}OW + WF = OF \\ \frac{UW}{OW} = tan ∠ UOW \\ \frac{WF}{UW - EF} = tan ∠ UED\end{matrix} \qquad ( 3 )$$

To solve:

$$WF = \frac{OF tan ∠ UOW - EF}{tan ∠ UOW + \frac{1}{tan ∠ UED}} \qquad ( 4 )$$

$$OW = OF - WF \qquad ( 5 )$$

$$UX = OW ( tan ∠ UOW + tan ∠ WOX ) \qquad ( 6 )$$

Assume UY≈UX, then [symbol] ≈UXtan∠ZUY, where [symbol] is the estimated length of the line segment ZY (i.e., between the corneal vertex Z and the line 17), and [symbol] is the estimated length of line segment YX.

According to analytic geometry, the following can be obtained:

$$\{ \begin{matrix}= tan ∠ WOX \\ = ( UX - ) tan ∠ ZUY\end{matrix} \qquad ( 7 )$$

Formula (7) is calculated iteratively until the error between two calculations of [symbol] is less than a predetermined or set value (e.g., an error set value), at which time the iteration ends, and the following is taken:

$$ZY = \qquad ( 8 )$$

The exophthalmia (e.g., the exophthalmic distance) is the distance ZG from the corneal vertex Z to the line DE:

$$ZG = WF + ZY \qquad ( 9 )$$

The exophthalmia is calculated according to formula (4) and formula (8).

It can be seen that in order to calculate the exophthalmia, the lengths of line segment YG and line segment ZY in FIG. 4 should be calculated first. Since the length of line segment YG is equal to the length of line segment WF, the exophthalmia can be calculated by the sum of the distances of line segment WF and line segment ZY.

The length of line segment WF and the distance of line segment UX can be obtained by solving the ternary first-order equation of formula (3). In Formula (7), it is initially assumed that UY is equal to UX, that is, YX is equal to zero. In this case, the estimated value of ZY can be solved by trigonometric functions, that is, [symbol] and the value of [symbol] can be calculated by [symbol] , and then the value of [symbol] can be calculated by [symbol] through iterative calculations until [symbol] converges. That is, when the error value calculated for [symbol] is less than the set or predetermined value on at least two occasions, then the iteration ends, that is, the length of line segment ZY is considered to be the final calculation result of [symbol] .

The above calculation process can be automatically calculated by computer programming. By obtaining the video frame having the longest reflected light from the visible light columns in the mirror, input to the computer, one can automatically output the exophthalmia evaluation.

### Example 4

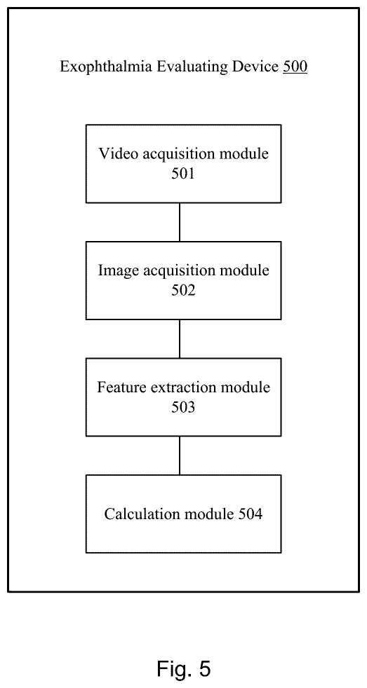

FIG. 5 is a structural diagram of an exophthalmia evaluation system 500 provided by an embodiment of the invention, as shown in FIG. 5. The system 500 comprises the following components.

A video acquisition module 501, configured to acquire the video of the eyeball looking straight ahead, illuminated by the near-infrared light source when the multiple visible light columns light up successively.

An image acquisition module 502, configured to obtain the video frame having the longest reflected light (from the visible light columns) in the mirror from the video or video frame.

A feature extraction module 503, configured to extract the corneal vertex and the pupil center from the video frame, as described herein.

A calculation module 504, configured to calculate the exophthalmia from a known position and the mirror tilt angle, the corneal vertex, and the pupil center, wherein the known position is the position of the optical center of the camera, the distance between the optical center of the camera and the video frame, and the position of the outer canthus of the corner of the eye, and the tilt angle of the mirror is the angle between the mirror and the imaging surface. The calculation module 504 may include a processor configured to retrieve and execute at least one program or program code that practices or implements the present method, as well as a computer-readable storage medium that stores the program(s) and/or program code.

In the above examples, a computer-readable storage medium stores at least one program or program code, and the program(s) and/or program code can be loaded and executed by a processor to perform a method of evaluating the exophthalmia according to the second aspect of the invention.

For example, the computer-readable storage medium can be a Read-Only Memory (ROM), a Random Access Memory (RAM), a Compact Disc Read-Only Memory (CDROM), a magnetic tape, a floppy disk or an optical data storage device.

A person of ordinary skill in the art may understand that all or part of the steps to implement the above embodiments may be performed by hardware or by hardware associated with at least one program code, which may be stored in a computer-readable storage medium, such as a read-only memory, a disk or an optical disc.

The above is only a description of embodiments of the invention, and is not intended to limit the invention, and any modification, equivalent replacement, improvement, etc. made within the spirit and principles of the invention shall be included in the scope of protection of the invention.

What is claimed is:

1. A device for evaluating exophthalmia in exophthalmia testing, comprising:
a left displacement platform,
a left canthal latch point,
a left mirror,
cameras,
a near-infrared light source,
a right canthal latch point,
a right mirror,
a right displacement platform,
visible light columns, and
a shell or housing, wherein:
the left mirror and the right mirror are respectively on the left canthal latch point and the right canthal latch point,
the left canthal latch point and the right canthal latch point are respectively on the left displacement platform and the right displacement platform,
the left displacement platform and the right displacement platform are respectively on opposite sides of the shell or housing,
the left mirror and the right mirror are angled,
the cameras are in front of the left mirror and the right mirror,
the near infrared light source is below the cameras, and
the visible light columns are configured longitudinally inside the shell or housing.

2. The device of claim 1, wherein the cameras comprise a first camera in front of the left mirror and a second camera in front of the right mirror.

3. The device of claim 1, wherein each of the visible light columns includes one or more discrete point light sources.

4. The device of claim 1, wherein each of the visible light columns is along an inner surface of the shell or housing.

5. The device of claim 1, further comprising a jaw support and a head rest, wherein each of the jaw support and the head rest are configured to place the eye in a proximity of an end of at least one of the left displacement platform and the right displacement platform.

6. The device of claim 1, wherein the left displacement platform and the right displacement platform are (i) adjustable along one or more Cartesian dimensions and (ii) configured to position a corresponding canthal latch point and/or mirror relative to the eye.

7. The device of claim 1, wherein the left mirror and the right mirror are respectively fixed to the left canthal latch point and the right canthal latch point, and the left canthal latch point and the right canthal latch point rotate angularly or move in one or more Cartesian dimensions to position the left mirror and the right mirror relative to the outer canthus.

8. A method for evaluating exophthalmia, comprising the following steps:
positioning an outer canthus of an eye,
emitting light from a near-infrared light source and emitting light successively from multiple visible light columns,
recording a video of an eyeball of the eye using a camera,
obtaining from the video a frame of the video having a longest reflected light from the visible light columns on the eyeball in a mirror;
determining a corneal vertex from the frame of the video;
determining a pupil center of the eyeball using a neural network; and
calculating the exophthalmia from known positions and a tilt angle of the mirror, the corneal vertex, and the pupil center; wherein:
the known positions comprise a position of an optical center of the camera, a distance between the optical center of the camera and the frame of the video, and a position of the outer canthus, and
the tilt angle of the mirror is an angle between the mirror and an imaging surface of the camera.

9. The method of claim 8, wherein the multiple visible light columns are along an inner surface of a shell or housing, and light is emitted successively from a middle of the shell or housing to an outside of the shell or housing, or from the outside of the shell or housing to the middle of the shell or housing.

10. The method of claim 9, wherein the video of the eyeball on one side of the shell or housing is captured when the multiple visible light columns on an opposite side of the shell or housing emit light.

11. The method of claim 8, wherein the exophthalmia is calculated by a process comprising:
defining the optical center of the camera as O, the outer canthus of the eye as E, the distance between the optical center O and the frame of the video as OB, a first imaging point of a corneal vertex of the eye on the frame of the video as an imaging point C, a second imaging point of a reflection point of the corneal vertex in the mirror on the frame of the video as an imaging point A, a vertical distance between the imaging point C and an optical axis of the camera as AB, a vertical distance between the imaging point A and the optical axis of the camera as CB, the tilt angle between the mirror and the eye as ∠UED, the distance between the optical center and a plane of the outer canthus perpendicular to the optical axis as OF, and a distance between the outer canthus and the optical axis as EF, and
calculating the exophthalmia as a distance between the corneal vertex and a straight line DE in the plane of the outer canthus that is parallel to a line that passes through the reflection point of the corneal vertex in the mirror and intersects with the optical axis at a point W, with a line ZG defining a length or distance of the exophthalmia at a point Y, and with an extension of a line between the optical center O and the imaging point C at a point X.

12. The method of claim 11, wherein the process for calculating the exophthalmia further comprises:
finding a length of a line segment WF according to:
$$WF = \frac{OF tan ∠ UOW - EF}{tan ∠ UOW + \frac{1}{tan ∠ UED}}$$
where <UOW is an angle between (i) a line from the reflection point to the optical center and (ii) the optical axis;
iteratively calculate a length of a line segment ZY from the corneal vertex to the point Y; and
calculating the length or distance of the exophthalmia according to ZG=WF+ZY.

13. The method of claim 8, wherein determining the pupil center of the eye comprises using the neural network to determine a location of the pupil center of the eye.

14. The method of claim 8, wherein positioning the outer canthus comprises positioning a head containing the eye in a jaw support and a head rest, wherein at least one of the jaw support and the head rest is configured to place the eye in proximity to an end of a canthal latch point to which the mirror is fixed or mounted.

15. The method of claim 14, further comprising adjusting a position of the displacement platform and/or the canthal latch point to bring the mirror to a predetermined position relative to a position of the outer canthus.

16. A system for evaluating exophthalmia, comprising:
a video acquisition module, configured to acquire a video of an eyeball looking straight ahead when a near infrared light source emits light towards the eyeball and multiple visible light columns successively emit light towards the eyeball;
an image acquisition module, configured to obtain a frame of the video having a longest reflected light from the video;
a feature extraction module, configured to extract the corneal vertex and the pupil center from the frame of the video; and
a calculation module, configured to calculate the exophthalmia from known positions and the mirror tilt angle, the corneal vertex, and the pupil center, wherein the known positions comprise a position of an optical center of a camera in the video acquisition module, a distance between the optical center of the camera and the frame of the video, and a position of an outer canthus of an eye including the eyeball.

17. A tangible computer-readable storage medium, storing at least one program code adapted to be loaded and executed by a processor to perform the method of claim 8.
